(19) United States
(12) Patent Application Publication
(10) Pub. No.: US 2007/0173456 A1
(43) Pub. Date: Jul. 26, 2007

(54) PREPTIN FRAGMENTS AND METHODS OF USE

(75) Inventors: Jillian Cornish, Auckland (NZ); Ian Reginald Reid, Auckland (NZ)

Correspondence Address: FISH & RICHARDSON PC, P.O. BOX 1022, MINNEAPOLIS, MN, 55440-1022, US

(73) Assignee: Auckland UniServices Limited, Auckland (NZ)

(21) Appl. No.: 11/608,380

(22) Filed: Dec. 8, 2006

Related U.S. Application Data
(60) Provisional application No. 60/748,945, filed on Dec. 9, 2005.

(51) Int. Cl. A61K 38/10; A61K 38/16; A61K 48/00

(52) U.S. Cl. 514/13; 514/14; 514/44

(57) ABSTRACT

Methods for increasing osteoblast proliferation with N-terminal fragments of preptin are provided herein.

### CROSS-REFERENCE TO RELATED APPLICATION

This application claims the benefit of priority of U.S. Provisional Patent Application No. 60/748,945, filed Dec. 9, 2005, the contents of which is hereby incorporated by reference in its entirety for all purposes.

### TECHNICAL FIELD

This invention relates to peptide compositions, methods of modulating osteoblasts, and bone disorders.

### BACKGROUND

Preptin is a 34-amino acid peptide corresponding to Asp⁶⁹-Leu¹⁰² of the proinsulin-like growth factor II. It is present in pancreatic islet beta cells and undergoes glucose-mediated co-secretion with insulin.

The two-phase secretion of insulin in response to glucose is well characterized. See, e.g., Grodsky et al., Acta Diabetol. Lat. 5: 140-161, 1968. The first phase results in a transient spike in insulin secretion, while the second phase results in a progressive increase in insulin release. Preptin has been found to enhance, but not initiate, insulin secretion. More specifically, infusion of preptin into the isolated, perfused rat pancreas exerts a significant increase in the second phase of secretion, while removal of preptin from the pancreas by adding anti-preptin antibodies results in a decrease in both the first and the second phases of insulin secretion. See Buchanan et al., Biochem. J. 360: 431-439, 2001. It also has been suggested that preptin elicits its effects by binding to a cell surface receptor (Buchanan supra).

### SUMMARY

The present invention is based, in part, on the unexpected finding that fragments of preptin can stimulate proliferation of osteoblasts, which play a critical role in bone growth. Accordingly, peptide compositions that include the fragments, and methods of using them, are provided herein. Also provided are nucleic acids encoding the peptides and uses thereof.

In one aspect, this invention features a method for treating a subject at risk for, or suffering from, a bone condition. The subject can be, e.g., a mammal, a human, a horse, a dog, or a cat. The method includes administering an effective amount of a peptide that includes an N-terminal fragment of preptin or a variant thereof (e.g., a variant which stimulates osteoblast growth, a variant which modulates osteoblast apoptosis, a variant which has a high affinity for a receptor expressed on an osteoblast, a variant which is a preptin agonist) to the subject. In various embodiments, the peptide includes between 5 and 20 consecutive amino acids of the 20 N-terminal amino acids of preptin, or a variant sequence thereof. For example, the peptide includes a sequence at least 80% identical to the following sequence: Asp Val Ser Thr R₁ R₂ R₃ Val Leu Pro Asp R₄ Phe Pro Arg Tyr Pro Val Gly Lys (SEQ ID NO:1)(preptin 1-20), wherein R₁ is Ser or Pro, R₂ is Gln or Pro, R₃ is Ala or Thr, and R₄ is Asp or Asn; or a fragment thereof with 5, 6, 7, 8, 9, 10, 11, 12, 13, 14, 15, 16, 17, 18, or 19 consecutive residues of SEQ ID NO:1.

Variant peptides may have, for example, deletions, insertions, or substitutions at 1, 2, 3, 4, 5, 6, or 7 amino acid positions of the sequence (but generally have substitutions at fewer than 30% of total residues of the sequence). Amino acid substitutions can be conservative or non-conservative.

The peptides for use in methods described herein are other than full-length preptin peptides (i.e., the peptides include fewer than 21 amino acids of the N-terminus of preptin). However, the peptides can include non-preptin (i.e., heterologous) amino acid sequences, and therefore may be longer than 20 amino acids. For example, a preptin fragment may be fused to a heterologous polypeptide to increase circulating half-life of the fragment in a subject. An example of a heterologous polypeptide that increases circulating half-life is an Fc portion of an immunoglobulin (e.g., Fc of an IgG molecule).

In one embodiment, the peptide has the following sequence: Asp Val Ser Thr R₁ R₂ R₃ Val Leu Pro Asp R₄ Phe Pro Arg Tyr Pro Val Gly (SEQ ID NO:7)(preptin 1-19), wherein R₁ is Ser or Pro, R₂ is Gln or Pro, R₃ is Ala or Thr, and R₄ is Asp or Asn.

In one embodiment, the peptide has the following sequence: Asp Val Ser Thr R₁ R₂ R₃ Val Leu Pro Asp R₄ Phe Pro Arg Tyr Pro Val (SEQ ID NO:8)(preptin 1-18), wherein R₁ is Ser or Pro, R₂ is Gln or Pro, R₃ is Ala or Thr, and R₄ is Asp or Asn.

In another embodiment, the peptide has the following sequence: Asp Val Ser Thr R₁ R₂ R₃ Val Leu Pro Asp R₄ Phe Pro Arg Tyr Pro (SEQ ID NO:9)(preptin 1-17), wherein R₁ is Ser or Pro, R₂ is Gln or Pro, R₃ is Ala or Thr, and R₄ is Asp or Asn.

In another embodiment, the peptide has the following sequence: Asp Val Ser Thr R₁ R₂ R₃ Val Leu Pro Asp R₄ Phe Pro Arg Tyr (SEQ ID NO:2)(preptin 1-16), wherein R₁ is Ser or Pro, R₂ is Gln or Pro, R₃ is Ala or Thr, and R₄ is Asp or Asn.

In yet another embodiment, the peptide has the following sequence: Asp Val Ser Thr R₁ R₂ R₃ Val Leu Pro Asp R₄ Phe Pro Arg (SEQ ID NO:10)(preptin 1-15), wherein R₁ is Ser or Pro, R₂ is Gln or Pro, R₃ is Ala or Thr, and R₄ is Asp or Asn.

The subject can be at risk for, or suffering from a disease associated with excessive resorption or breakdown of bone tissue, a decrease in bone formation, or decreased bone density. Examples of such diseases include, but are not limited to, osteoporosis, osteopenia, bone defects, and osteogenesis imperfecta. The subject can also be at risk for, or suffering from bone loss as a result of immobility, bone fractures, malignancy, primary hyperparathyroidism, endocrine disorders, autoimmune arthritis, or drug use (e.g., steroid drugs). The subject can also be undergoing a treatment (e.g., corticosteroid treatment, bone marrow transplantation, or oophorectomy) known to result in bone loss. The term “bone condition” refers to any disease or symptom wherein mediation of osteoblast or osteoclast activity (or levels) is involved, and includes any of the diseases or situations described above.

Also provided herein are methods for treating a subject at risk for, or suffering from, a bone condition, by administering to the subject a nucleic acid encoding any of the peptides described herein.

In another aspect, the invention features methods for stimulating osteoblast growth in a subject, e.g., a mammal, a human, a horse, a dog, or a cat. The subject can be a subject identified in need of stimulation of osteoblast activity. The methods include, for example, administering an amount of a peptide effective to stimulate osteoblast proliferation in the subject. The peptide includes an N-terminal fragment of preptin or a variant thereof (e.g., a variant which stimulates osteoblast growth, a variant which modulates osteoblast apoptosis, a variant which has a high affinity for a receptor expressed on an osteoblast, a variant which is a preptin agonist) described herein. In various embodiments, the peptide includes between 5 and 20 consecutive amino acids of the 20 N-terminal amino acids of preptin, or a variant sequence thereof. For example, the peptide includes a sequence at least 80% identical to the following sequence: Asp Val Ser Thr R₁ R₂ R₃ Val Leu Pro Asp R₄ Phe Pro Arg Tyr Pro Val Gly Lys (SEQ ID NO:1), wherein R₁ is Ser or Pro, R₂ is Gln or Pro, R₃ is Ala or Thr, and R₄ is Asp or Asn; or a fragment thereof with 5, 6, 7, 8, 9, 10, 11, 12, 13, 14, 15, 16, 17, 18, or 19 consecutive residues of SEQ ID NO:1.

Variant peptides may have, for example, deletions, insertions, or substitutions at 1, 2, 3, 4, 5, 6, or 7 amino acid positions of the preptin sequence (but generally have substitutions at fewer than 30% of total residues of the preptin sequence). Amino acid substitutions can be conservative or non-conservative.

In one embodiment, the peptide is a preptin fragment fused to a heterologous polypeptide. In one embodiment, the heterologous polypeptide increases circulating half-life of the fragment in a subject.

In one embodiment, the peptide has the following sequence: Asp Val Ser Thr R₁ R₂ R₃ Val Leu Pro Asp R₄ Phe Pro Arg Tyr Pro Val Gly (SEQ ID NO:7), wherein R₁ is Ser or Pro, R₂ is Gln or Pro, R₃ is Ala or Thr, and R₄ is Asp or Asn.

In one embodiment, the peptide has the following sequence: Asp Val Ser Thr R₁ R₂ R₃ Val Leu Pro Asp R₄ Phe Pro Arg Tyr Pro Val (SEQ ID NO:8), wherein R₁ is Ser or Pro, R₂ is Gln or Pro, R₃ is Ala or Thr, and R₄ is Asp or Asn.

In another embodiment, the peptide has the following sequence: Asp Val Ser Thr R₁ R₂ R₃ Val Leu Pro Asp R₄ Phe Pro Arg Tyr Pro (SEQ ID NO:9), wherein R₁ is Ser or Pro, R₂ is Gln or Pro, R₃ is Ala or Thr, and R₄ is Asp or Asn.

In another embodiment, the peptide has the following sequence: Asp Val Ser Thr R₁ R₂ R₃ Val Leu Pro Asp R₄ Phe Pro Arg Tyr (SEQ ID NO:2), wherein R₁ is Ser or Pro, R₂ is Gln or Pro, R₃ is Ala or Thr, and R₄ is Asp or Asn.

In yet another embodiment, the peptide has the following sequence: Asp Val Ser Thr R₁ R₂ R₃ Val Leu Pro Asp R₄ Phe Pro Arg (SEQ ID NO:10), wherein R₁ is Ser or Pro, R₂ is Gln or Pro, R₃ is Ala or Thr, and R₄ is Asp or Asn.

Also provided are methods for stimulating osteoblast growth in a subject by administering to the subject a nucleic acid encoding any of the peptides described herein.

In another aspect, the invention features methods for increasing proliferation of a cell, e.g., a cell that expresses a receptor for preptin, such as an osteoblast. The method includes contacting the cell with a peptide containing an N-terminal fragment of preptin, or a variant thereof (e.g., a variant which stimulates osteoblast growth, a variant which modulates osteoblast apoptosis, a variant which has a high affinity for a receptor expressed on an osteoblast, a variant which is a preptin agonist), under conditions in which the peptide binds to the cell. The cell is contacted with the peptide in vitro or in vivo.

The peptide is a peptide described herein, e.g., the peptide includes between 5 and 20 consecutive amino acids of the 20 N-terminal amino acids of preptin, or a variant sequence thereof. For example, the peptide includes a sequence at least 80% identical to the following sequence: Asp Val Ser Thr R₁ R₂ R₃ Val Leu Pro Asp R₄ Phe Pro Arg Tyr Pro Val Gly Lys (SEQ ID NO:1), wherein R₁ is Ser or Pro, R₂ is Gln or Pro, R₃ is Ala or Thr, and R₄ is Asp or Asn; or a fragment thereof with 5, 6, 7, 8, 9, 10, 11, 12, 13, 14, 15, 16, 17, 18, or 19 consecutive residues of SEQ ID NO:1.

Variant peptides have, for example, deletions, insertions, or substitutions at 1, 2, 3, 4, 5, 6, or 7 amino acid positions of the preptin sequence (but generally have substitutions at fewer than 30% of total residues of the preptin sequence). Amino acid substitutions can be conservative or non-conservative.

In one embodiment, the peptide is a preptin fragment fused to a heterologous polypeptide. In one embodiment, the heterologous polypeptide increases circulating half-life of the fragment in a subject.

In one embodiment, the peptide has the following sequence: Asp Val Ser Thr R₁ R₂ R₃ Val Leu Pro Asp R₄ Phe Pro Arg Tyr Pro Val Gly (SEQ ID NO:7), wherein R₁ is Ser or Pro, R₂ is Gln or Pro, R₃ is Ala or Thr, and R₄ is Asp or Asn.

In one embodiment, the peptide has the following sequence: Asp Val Ser Thr R₁ R₂ R₃ Val Leu Pro Asp R₄ Phe Pro Arg Tyr Pro Val (SEQ ID NO:8), wherein R₁ is Ser or Pro, R₂ is Gln or Pro, R₃ is Ala or Thr, and R₄ is Asp or Asn.

In another embodiment, the peptide has the following sequence: Asp Val Ser Thr R₁ R₂ R₃ Val Leu Pro Asp R₄ Phe Pro Arg Tyr Pro (SEQ ID NO:9), wherein R₁ is Ser or Pro, R₂ is Gln or Pro, R₃ is Ala or Thr, and R₄ is Asp or Asn.

In another embodiment, the peptide has the following sequence: Asp Val Ser Thr R₁ R₂ R₃ Val Leu Pro Asp R₄ Phe Pro Arg Tyr (SEQ ID NO:2), wherein R₁ is Ser or Pro, R₂ is Gln or Pro, R₃ is Ala or Thr, and R₄ is Asp or Asn.

In yet another embodiment, the peptide has the following sequence: Asp Val Ser Thr R₁ R₂ R₃ Val Leu Pro Asp R₄ Phe Pro Arg (SEQ ID NO:10), wherein R₁ is Ser or Pro, R₂ is Gln or Pro, R₃ is Ala or Thr, and R₄ is Asp or Asn.

This invention also features an article of manufacture that includes a vessel containing the peptides (e.g., peptides that include N-terminal fragments of preptin); and instructions for use of the peptides for treatment of a bone condition by administering an effective amount of the peptide to a subject. Articles of manufacture containing nucleic acids encoding the peptides are also provided.

Also within the scope of this invention is an article of manufacture. The article includes packaging material; and contained within the packaging material, a peptide described herein. The packaging material comprises a label that indicates that the peptide can be used for treating a bone condition (e.g., osteoporosis, osteopenia, bone defects, or osteogenesis imperfecta) in a subject. In other aspects, the label includes dosage information. Also provided are articles of manufacture containing nucleic acids encoding the peptides.

As used herein, “preptin” is an isolated peptide of 34 amino acids in length with the following sequence: Asp Val Ser Thr R₁ R₂ R₃ Val Leu Pro Asp R₄ Phe Pro Arg Tyr Pro Val Gly Lys Phe Phe R₅ R₆ Asp Thr Trp R₇ Gln Ser R₈ Rg Arg Leu (SEQ ID NO:3), wherein: R₁ is Ser or Pro; R₂ is Gln or Pro; R₃ is Ala or Thr; R₄ is Asp or Asn; R₅ is Gln or Lys; R₆ is Tyr or Phe; R₇ is Arg or Lys; R₈ is Ala or Thr, and R₉ is Gly or Gln. It includes mouse preptin, rat preptin, and human preptin, the sequences of which are shown below.

(SEQ ID NO:4)

| | |
|---|---|
| Mouse preptin: | DVSTSQAVLPDDFPRYPVGKFFQYDTWRQSAGRL |

(SEQ ID NO: 5)

| | |
|---|---|
| Rat preptin: | DVSTSQAVLPDDFPRYPVGKFFKFDTWRQSAGRL |

(SEQ ID NO: 6)

| | |
|---|---|
| Human preptin: | DVSTPPTVLPDNFPRYPVGKFFQYDTWKQSTQRL |

The amino acid sequences correspond to Asp₆₉-Leu₁₀₂ of the proIGF-II E-peptide in each mammal.

In various embodiments, the peptides described herein have preptin agonist activity. “A preptin agonist” is a compound which (1) has a high affinity (e.g., a Ki of 10⁻⁷-10⁻⁹ M, a Ki of 10⁻⁸-10⁻⁹ M) for a preptin-binding receptor (as defined by the receptor binding assay described in Motulsky, H. J and Mahan, L. C., Mol. Pharmacol. 25: 1, 1984; and (2) promotes the proliferation of bone cells, e.g., osteoblasts.

The “percent identity” of two amino acid sequences can be determined using the algorithm of Karlin and Altschul (1990, Proc. Natl. Acad. Sci. USA 87: 2264-2268), modified as in Karlin and Altschul (1993, Proc. Natl. Acad. Sci. USA 90: 5873-5877). Such an algorithm is incorporated into the NBLAST and XBLAST programs (version 2.0) of Altschul et al. (1990) J. Mol. Biol. 215: 403-10. BLAST protein searches can be performed with the XBLAST program, score=50, wordlength=3 to obtain amino acid sequences homologous to the peptide molecules described herein. Where gaps exist between two sequences, Gapped BLAST can be utilized as described in Altschul et al. (1997) Nucleic Acids Res. 25(17): 3389-3402. When utilizing BLAST and Gapped BLAST programs, the default parameters of the respective programs (e.g., XBLAST and NBLAST) can be used.

A “conservative amino acid substitution” is one in which an amino acid residue is replaced with another residue having a chemically similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid analogs (e.g., phosphorylated amino acids) are also contemplated in the present invention.

As used herein, a subject may have substantially normal bone density or may be at risk of bone deterioration. Examples of the latter type of subjects include postmenopausal women, usually at age 50 and over, and men over 60 years of age.

The term “osteoblast” refers to bone-forming cells.

The methods herein include administering to a subject (including a subject identified as in need of such treatment, e.g., a subject in need of modulation of osteoblast activity) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. All cited patents, patent applications, and references (including references to public sequence database entries) are incorporated by reference in their entireties for all purposes. U.S. Provisional App. No. 60/748,945, filed Dec. 9, 2005, is incorporated by reference in its entirety for all purposes.

The details of one or more embodiments of the invention are set forth in the description below. Other features, objects, and advantages of the invention will be apparent from the description and from the claims.

### DESCRIPTION OF DRAWINGS

FIG. 1 is a bar graph depicting the effects of full-length preptin (preptin 1-34), an N-terminal fragment of preptin (preptin 1-19), a C-terminal fragment of preptin (preptin 20-34), and vehicle alone (control) on proliferation of rat osteoblasts, as measured by thymidine incorporation. * represents p<0.001 (significantly different from control). “T/C” refers to treatment to control ratios.

FIG. 2 is a bar graph depicting the effects of various concentrations of an N-terminal fragment of preptin (preptin 1-16), or vehicle (control) on proliferation of rat osteoblasts, as measured by thymidine incorporation.

FIG. 3 is a bar graph depicting the effects of various concentrations of an N-terminal fragment of preptin (preptin 1-16), or vehicle (control) on proliferation of rat osteoblasts, as measured by cell number.

FIG. 4 is a bar graph depicting the effects of various concentrations of preptin 1-16 (16PRP), preptin 1-19 (nPRP), a C-terminal fragment of preptin (cPRP; preptin 20-34), and vehicle alone (control) on proliferation of rat osteoblasts, as measured by thymidine incorporation.

### DETAILED DESCRIPTION

This invention relates to use of N-terminal fragments of preptin, and analogs thereof, for stimulating osteoblast growth or modulating osteoblast apoptosis. In various embodiments, the fragments and analogs thereof are fragments which do not have activity in the pancreas (e.g., the fragments and analogs thereof do not effect carbohydrate metabolism), unlike full-length preptin.

Peptide Agents

Fragments of preptin, and variants thereof, can be prepared by peptide synthesis or by recombinant methods method. Synthesis of peptides is well established in the art. See, e.g., Stewart et al., Solid Phase Peptide Synthesis (2^{nd} Ed.), 1984; and Chan, “Fmoc Solid Phase Peptide Synthesis, A Practical Approach,” Oxford University Press, 2000. The peptides may be synthesized using an automated peptide synthesizer (e.g., a Pioneer™ Peptide Synthesizer, Applied Biosystems, Foster City, Calif.). For example, a peptide is prepared on methylbenzyhydrylamine resin followed by hydrogen fluoride deprotection and cleavage from the resin. The synthesized peptide can be further purified by a method such as affinity column chromatography or high pressure liquid chromatography. Standard physicochemical characterization techniques are known in the art, including NMR (¹³C, ¹H, ¹⁹F, or ³¹P) and IR, which can provide confirmatory evidence of the identity and purity of the synthetic products. Amino acid analysis can also be used to confirm the amino acid composition of the peptide. Mass spectroscopy can be used to identify the molecular weight of synthetic products.

In various embodiments, the peptides (e.g., N-terminal fragments of preptin, such as preptin 1-16) are modified to increase stability. These modified peptides include, for example, analogs containing one or more non-peptide bonds (which replace the peptide bonds), non-naturally occurring amino acids, and cyclic analogs. Included herein are analogs in which one or more of the amino acids are D amino acids. These types of analogs may have increased stability or activity. See, e.g., Chover et al., Acc. Chem. Res. 26:266-273, 1993; Goodman et al., Acc. Chem. Res. 12:1-7, 1979.

Peptides (e.g., N-terminal fragments of preptin, variants, and analogs thereof) can be tested for their abilities to stimulate osteoblast growth or modulate osteoblast apoptosis by examining their activities in the in vitro assays described herein. See the specific examples below. In vivo screening can also be performed by following procedures well known in the art. See, e.g., Cornish et al., Am J Physiol 273: E1113-E1120, 1997; and Cornish et al., Am J Physiol 279: E730-E735, 2000, which is hereby incorporated by reference.

Nucleic Acid Agents

The peptides described herein can be encoded by a nucleic acid molecule, including a molecule of genomic DNA, cDNA, or synthetic DNA, and such nucleic acids are within the scope of the present invention. The nucleic acids may be formulated in a manner that can be described as substantially pure. For example, a substantially pure nucleic acid molecule of the invention can be separated from other nucleic acid molecules and/or separated from other biological molecules.

The sequences of the nucleic acid molecules can vary due to the degenerate nature of the genetic code, and degenerate variants are within the scope of the present invention.

The nucleic acid molecules encoding a peptide described herein (e.g., an N-terminal fragment of preptin such as preptin 1-16) may be contained within a vector that is capable of directing expression of the peptide in, for example, a cell that contains the vector (e.g., a cell that has been transduced with the vector). The vectors can be viral vectors (e.g., a retroviral, adenoviral, or adenoviral-associated vector), as well as plasmids or cosmids. Suitable vectors include T7-based vectors for use in bacteria (see, e.g., Rosenberg et al., Gene, 56:125, 1987), the pMSXND expression vector for use in mammalian cells (Lee and Nathans, J. Biol. Chem., 263:3521, 1988), and baculovirus-derived vectors (for example, the expression vector pBacPAK9 from Clontech, Palo Alto, Calif., USA) for use in insect cells. While additional promoters are described elsewhere, we note that a T7 promoter can be used when the host cells are bacterial, and a polyhedron promoter can be used in insect cells.

Yeast vectors typically contain an origin of replication sequence, an autonomously replicating sequence (ARS), a promoter region, sequences for polyadenylation, sequences for transcription termination, and a selectable marker. Suitable promoter sequences for yeast vectors include promoters for metallothionein, 3-phosphoglycerate kinase (Hitzeman et al., J. Biol. Chem., 255:2073, 1980) or other glycolytic enzymes (Hess et al., J. Adv. Enzyme Reg., 7:149, 1968; and Holland et al., Biochem., 17:4900, 1978), such as enolase, glyceraldehyde-3-phosphate dehydrogenase, hexokinase, pyruvate decarboxylase, phosphofructokinase, glucose-6phosphate isomerase, 3-phosphoglycerate mutase, pyruvate kinase, triosephosphate isomerase, phosphoglucose isomerase, and glucokinase. Other suitable vectors and promoters for use in yeast expression are further described in Hitzeman, EP-A-73,657.

Mammalian expression vectors typically include nontranscribed regulatory elements such as an origin of replication, a promoter sequence, an enhancer linked to the structural gene, other 5′ or 3′ flanking nontranscribed sequences (e.g., ribosome binding sites, a polyadenylation site, splice donor and acceptor sites, and transcriptional termination sequences). Regulatory sequences derived from Polyoma, Adenovirus 2, Simian Virus 40 (SV40), and human cytomegalovirus are frequently used for recombinant expression in mammalian cells. For example, SV40 origin, early and late promoter, enhancer, splice, and polyadenylation sites may be used to provide the other genetic elements required for expression of preptin DNA sequence in a mammalian host cell. Cytomegalovirus or metallothionein promoters are also frequently used in mammalian cells.

Prokaryotic (e.g., bacterial cells such as E. coli cells) or eukaryotic cells (e.g., yeast cells, or mammalian cells such as CHO cells) that contain and express nucleic acids encoding any of the peptides are also featured herein. For yeast expression, cells of the Saccharomyces genus (e.g., S. cerevisiae) may be used. Alternatively, cells of other genera of yeast, such as Pichia or Kluyveromyces, may be used.

Examples of suitable mammalian host cell lines for production of the peptides include: CHO cells; COS cell lines derived from monkey kidney, (e.g., COS-7 cells, ATCC number CRL 1651); L cells; C127 cells; 3T3 cells (ATCC number CCL 163); HeLa cells (ATCC number CCL 2); and BHK (ATCC number CRL 10) cell lines.

The method of transduction, the choice of expression vector, and the host cell may vary. The components of the expression system are compatible with one another, a determination that is well within the abilities of skilled artisans. Furthermore, for guidance in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1987).

Methods of Treatment and Pharmaceutical Compositions

Methods for treating bone conditions with effective amounts of peptides derived from the N-terminus of preptin, or nucleic acids encoding the peptides, are provided herein. Also provided are methods for increasing or maintaining bone density with the peptides, or nucleic acids encoding the peptides. The term “treating” is defined as the application or administration of a composition (e.g., a peptide including a fragment of preptin (e.g., a peptide which is not a full-length form of preptin), or a nucleic acid encoding the peptide), to a subject, who has, or is determined to have, a bone condition, a symptom of a bone condition, a disease or disorder secondary to a bone condition, or a predisposition toward a bone condition, with the purpose to cure, alleviate, relieve, remedy, or ameliorate the bone condition, the symptom of the bone condition, the disease or disorder secondary to the bone condition, or the predisposition toward the bone condition. “An effective amount” refers to an amount of the peptide or nucleic acid that confers a therapeutic effect on the treated subject. The therapeutic effect may be objective (i.e., measurable by some test or marker) or subjective (i.e., subject gives an indication of or feels an effect). An effective amount of the peptide or nucleic acid described above may range from about 1 μg/Kg body weight to about 1000 μg/Kg body weight. Effective doses will also vary depending on the route of administration, as well as the possibility of co-usage with other agents for stimulating osteoblast growth or modulating osteoblast apoptosis, such as a bone anti-resorptive agent (e.g., calcitonin or bisphosphonate) or a bone anabolic agent (e.g., parathyroid hormone, parathyroid hormone related protein, cytokines, or growth hormone).

As used herein, the peptides or nucleic acids are defined to include pharmaceutically acceptable derivatives (e.g., salts).

Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate, trifluoroacetate, and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

Also within the scope of this invention is a pharmaceutical composition that contains an effective amount of a peptide or nucleic acid described herein, and a pharmaceutically acceptable carrier.

The term “pharmaceutically acceptable carrier” refers to a carrier (adjuvant or vehicle) that may be administered to a patient, together with the peptide or nucleic acid, and which does not destroy the pharmacological activity thereof and is nontoxic when administered in doses sufficient to deliver the peptide or nucleic acid.

Pharmaceutically acceptable carriers that may be used in the pharmaceutical compositions described above include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein. Oil solutions or suspensions may also contain a long-chain alcohol diluent or dispersant, or carboxymethyl cellulose or similar dispersing agents, which are commonly used in the formulation of pharmaceutically acceptable dosage forms such as emulsions and or suspensions.

To practice the method for treating a bone condition or the method for increasing or maintaining bone density, a peptide described herein (e.g., a peptide including an N-terminal fragment of preptin), or a nucleic acid encoding the peptide, can be administered to a patient or a subject. The peptide or nucleic acid can, for example, be administered in a pharmaceutically acceptable carrier such as physiological saline, in combination with other drugs, and/or together with appropriate excipients. It also can, for example, be administered by injection, intravenously, intraarterially, subdermally, intraperitoneally, intramuscularly, or subcutaneously; or orally, buccally, nasally, transmucosally, topically, in an ophthalmic preparation, by inhalation, by intracranial injection or infusion techniques. The methods herein contemplate administration of an effective amount of compound or compound composition to achieve the desired or stated effect. Lower or higher doses than those described above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

A pharmaceutical composition may be orally administered in any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

A sterile injectable composition (e.g., aqueous or oleaginous suspension) can be formulated according to techniques known in the art using suitable dispersing or wetting agents (such as, for example, Tween 80) and suspending agents.

Topical administration of a pharmaceutical composition is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-applied transdermal patches are also included in this invention.

A pharmaceutical composition may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The peptides and nucleic acids described herein can be administered using agents or devices that allow for sustained or controlled release. For example, a peptide (e.g., a peptide which is an N-terminal fragment of preptin) can be administered with a biocompatible polymer, microparticle, or mesh, to reduce degradation and delay release of the peptide in a subject. These controlled release systems can be administered, e.g., by implantation (e.g., intramuscularly, subcutaneously, intravenously, or at an organ or joint cavity) or injection.

In some embodiments, a peptide described herein (e.g., an N-terminal fragment of preptin) is conjugated to a water-soluble polymer, e.g., to increase stability or circulating half life or reduce immunogenicity. Clinically acceptable, water-soluble polymers include, but are not limited to, polyethylene glycol (PEG), polyethylene glycol propionaldehyde, carboxymethylcellulose, dextran, polyvinyl alcohol (PVA), polyvinylpyrrolidone (PVP), polypropylene glycol homopolymers (PPG), polyoxyethylated polyols (POG) (e.g., glycerol) and other polyoxyethylated polyols, polyoxyethylated sorbitol, or polyoxyethylated glucose, and other carbohydrate polymers. Methods for conjugating polypeptides to water-soluble polymers such as PEG are described, e.g., in U.S. Pat. Pub. No. 20050106148 and references cited therein.

Other methods of controlled delivery of the peptide involve adding a second component or carrier to the active agent, typically in the form of a coating such that the coating acts to delay the release of the active agent in vivo (see, for example U.S. Pat. Nos. 4,060,598; 3,538,214; and 4,177,255). The active agent can also be dispersed in a gel formed from a monoglyceride and at least one vegetable oil, in amounts sufficient to form a reverse hexagonal liquid crystalline phase when in contact with an aqueous liquid (see, e.g., U.S. Pat. No. 5,143,934). Also useful are porous polymeric microparticles having preformed pores into which active agent is loaded (see, e.g., U.S. Pat. No. 5,470,582).

The nucleic acid molecules described herein (e.g., nucleic acid molecules encoding N-terminal fragments of preptin) can be inserted into vectors and used for expression in cells. Vecotrs can be delivered to a subject by, for example, intravenous injection, local administration (see U.S. Pat. No. 5,328,470) or by stereotactic injection (see e.g., Chen et al., PNAS 91:3054-3057, 1994). The pharmaceutical preparation of the vector can include the vector in an acceptable diluent, or can include a slow release matrix in which the gene delivery vehicle is imbedded. Alternatively, where the complete vector can be produced intact from recombinant cells, e.g. retroviral vectors, the pharmaceutical preparation can include one or more cells which produce the gene delivery system.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

The invention will be further described in the following examples. It should be understood that these examples are for illustrative purposes only and are not to be construed as limiting this invention in any manner.

### EXAMPLE 1

### N-Terminal Fragments of Preptin Stimulate Osteoblast Proliferation

Osteoblasts were isolated from 20 day fetal rat calvariae as previously described (Cornish et al., American Journal of Physiology—Endocrinology & Metabolism 277: E779-E783, 1999). Briefly, calvariae were dissected aseptically and the frontal and parietal bones are stripped of their periosteum. Only the central portions of the bones, free from suture tissue, were collected. The calvariae were treated twice with phosphate buffered saline (PBS) containing 3 mM EDTA (pH 7.4) for 15 minutes at 37° C. in a shaking water bath. Calvariae were washed once in PBS and treated twice with 3 ml of collagenase (1 mg/ml) for 7 minutes at 37° C. After discarding supernatants from the first two collagenase digestions, calvariae were treated twice with 3 ml collagenase (2 mg/ml) for 30 minutes at 37° C. The supernatants from these third and fourth collagenase digestions were collected, pooled and centrifuged. The pelleted cells were washed in Dulbecco's modified Eagle's medium (DME) with 10% fetal calf serum (FCS), resuspended in DME/10% FCS, and placed in 75 cm² flasks. Cells were incubated under 5% CO₂ and 95% air at 37° C. Cells were grown to confluence (approximately 5-6 days) and then subcultured.

To set up subcultures, cells were trypsinized in trypsin-EDTA (0.05%/0.53 mM), rinsed in minimal essential medium (MEM) with 5% FCS, resuspended in fresh medium, and seeded at 5×10⁴ cells/ml in 24-well plates (0.5 ml cell suspension per well, i.e., 1.4×10⁴ cells/cm²). The osteoblast-like character of these cells has been established by demonstration of high levels of alkaline phosphatase activity and osteocalcin production (Groot et al., Cell Biol. Int. Rep., 9:528, 1985), and a sensitive adenylate cylcase response to parathyroid hormone and prostaglandins (Herrmann-Erlee et al., IX Int. Conf. on Calcium Regulating Hormones and Bone Metabolism, 409, 1986).

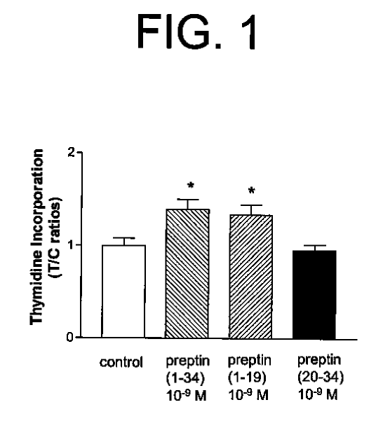

To evaluate the proliferative responses of osteoblast cultures in the presence of preptin and various fragments of preptin, subconfluent cell cultures were growth arrested by incubation in MEM/0.1% bovine serum albumin 24 hours prior to addition of test compounds. Cells were cultured in the presence of full-length preptin (preptin 1-34), preptin 1-19, preptin 20-34, or in the absence of preptin compounds (control). Preptin compounds were added to a final concentration of 1×10⁻⁹ M. A pulse of [³H]-thymidine (1 ∞Ci/well) was added to each sample two hours prior to the end of each experimental incubation. The incubations were terminated 20-24 hours after addition of preptin compounds by washing the cells in MEM containing non-radioactive thymidine, followed by the addition of 10% trichloroacetic acid. The precipitate was washed twice either ethanol:ether (3:1) and the wells were desiccated at room temperature. The residue in each well was redissolved in 2 M KOH at 55° C. for 30 minutes, neutralized with 1 M HCl, and an aliquot was removed to quantitate incorporation of radioactivity in the samples. The results are depicted in FIG. 1. These results reflect an average from at least six individual samples (wells) for each condition in a given assay (i.e., 6 wells for each of preptin 1-34, preptin 1-19, preptin 20-34, and control samples). The results are representative of at least four independent assays. As shown in FIG. 1, preptin 1-19 enhances osteoblast proliferation in vitro, to levels comparable to those stimulated by full-length preptin. No enhancement of osteoblast proliferation was observed with cells incubated with preptin 20-34.

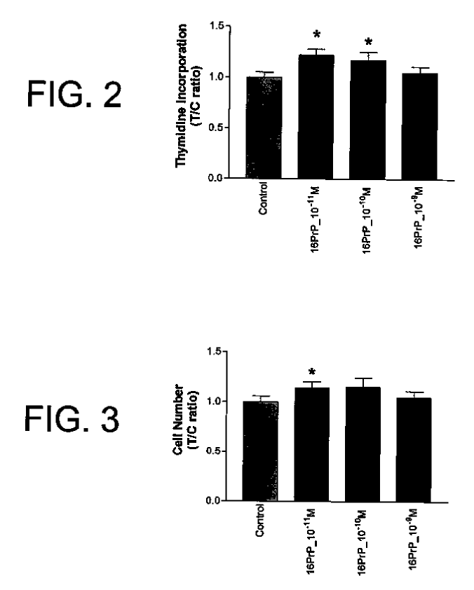

Next, a shorter N-terminal fragment of preptin, preptin 1-16, was tested for stimulation of osteoblast proliferation. Osteoblast proliferation was performed with primary osteoblast cultures as described above. Preptin 1-16 was tested at 1×10⁻⁹, 1×10⁻¹⁰, and 1×10⁻¹¹ M. The results are depicted in FIGS. 2 and 3, which show that preptin 1-16 stimulated proliferation at 1×10⁻¹⁰, and 1×10⁻¹¹ M.

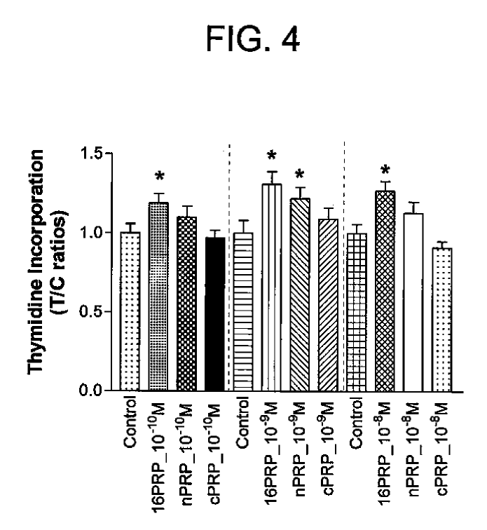

Next, stimulation of osteoblast proliferation by various concentrations of preptin 1-16 was compared in parallel with preptin 1-19 (nPRP) and preptin 20-34 (cPRP). Assays were performed as described above. The results are depicted in FIG. 4. Preptin 1-16 stimulated a significant increase in proliferation at all concentrations tested. Preptin 1-19 stimulated a significant increase in proliferation at 1×10⁻⁹M. Preptin 20-34 did not stimulate a significant increase in proliferation at any of the concentrations tested.

### EXAMPLE 2

### Promoting Bone Growth in Vivo

The effects of a preptin fragment (e.g., preptin 1-16, preptin 1-19) on bone growth can be determined in vivo as follows. Three groups of sexually mature male mice are given twice daily subcutaneous injections over the right hemicalvaria for 5 consecutive days. Two groups receive one of 2 doses of preptin 1-16 (0.825 μg or 8.25 μg per injection), and a further group receives vehicle. The animals are sacrificed 1 week after the last injection. Fluorochrome labels are injected subcutaneously at the base of the tail on days 1 (calcein), 5 (alizarin red) and 14 (calcein) to measure dynamic histomorphometric indices (such as, extent of mineralizing surface). Calvariae are excised, fixed in 10% neutral-buffered formalin, dehydrated and embedded in methylmethacrylate resin. Sections are cut, mounted on gelatin-coated slides, and histomorphometric indices (e.g., bone area and percentage of mineralizing surface of bone) are measured using image analysis. A significant increase in bone area and percentage of mineralizing surface of bone per total bone area observed in animals treated with preptin 1-16 as compared to animals treated with vehicle alone indicates that preptin 1-16 enhances bone growth in vivo.

### OTHER EMBODIMENTS

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a subject at risk for or suffering from a bone condition, the method comprising administering to the subject an effective amount of a peptide at least 80% identical to peptide of the following sequence: Asp Val Ser Thr R₁ R₂ R₃ Val Leu Pro Asp R₄ Phe Pro Arg Tyr Pro Val Gly Lys (SEQ ID NO:1), wherein R₁ is Ser or Pro, R₂ is Gln or Pro, R₃ is Ala or Thr, and R₄ is Asp or Asn; or a fragment thereof having between 5-19 consecutive amino acids of SEQ ID NO:1.

2. The method of claim 1, wherein the peptide is at least 90% identical to SEQ ID NO:1, or the fragment thereof.

3. The method of claim 1, wherein the peptide consists of SEQ ID NO:1.

4. The method of claim 1, wherein the peptide consists of 19 consecutive amino acids of SEQ ID NO:1.

5. The method of claim 1, wherein the peptide consists of 16 consecutive amino acids of SEQ ID NO:1.

6. The method of claim 5, wherein the peptide consists of the following sequence: Asp Val Ser Thr R₁ R₂ R₃ Val Leu Pro Asp R₄ Phe Pro Arg Tyr (SEQ ID NO:2), wherein R₁ is Ser or Pro, R₂ is Gln or Pro, R₃ is Ala or Thr, and R₄ is Asp or Asn.

7. The method of claim 6, wherein R₁ is Pro, R₂ is Pro, R₃ is Thr, and R₄ is Asn.

8. A method for stimulating osteoblast growth in a subject, the method comprising administering to the subject an effective amount of a peptide at least 80% identical to SEQ ID NO:1, or a fragment thereof having between 5-19 consecutive amino acids of SEQ ID NO:1.

9. The method of claim 8, wherein the method comprises identifying the subject as in need of stimulation of osteoblast growth, prior to the administering.

10. The method of claim 8, wherein the peptide is at least 90% identical to SEQ ID NO:1, or the fragment thereof.

11. The method of claim 8, wherein the peptide consists of SEQ ID NO:1.

12. The method of claim 8, wherein the peptide consists of 19 consecutive amino acids of SEQ ID NO:1.

13. The method of claim 8, wherein the peptide consists of 16 consecutive amino acids of SEQ ID NO:1.

14. The method of claim 13, wherein the peptide consists of the following sequence: Asp Val Ser Thr R₁ R₂ R₃ Val Leu Pro Asp R₄ Phe Pro Arg Tyr (SEQ ID NO:2), wherein R₁ is Ser or Pro, R₂ is Gln or Pro, R₃ is Ala or Thr, and R₄ is Asp or Asn.

15. The method of claim 14, wherein R₁ is Pro, R₂ is Pro, R₃ is Thr, and R₄ is Asn.

16. A method for increasing proliferation of a cell that expresses a receptor for preptin, the method comprising:
contacting the cell with a peptide under conditions in which the peptide binds to the cell, wherein the peptide is at least 80% identical to SEQ ID NO:1, or a fragment thereof having between 5-19 consecutive amino acids of SEQ ID NO:1, thereby increasing proliferation of the cell.

17. The method of claim 16, wherein the cell is an osteoblast.

18. A pharmaceutical composition comprising: a peptide at least 80% identical to SEQ ID NO:1, or a fragment thereof having between 5-19 consecutive amino acids of SEQ ID NO:1, wherein the peptide or fragment thereof stimulates osteoblast proliferation; and a pharmaceutically acceptable carrier.

19. A method for treating a subject at risk for or suffering from a bone condition, the method comprising administering to the subject a composition comprising a nucleic acid, wherein the nucleic acid comprises a sequence encoding a peptide at least 80% identical to SEQ ID NO:1, or a fragment thereof having between 5-19 consecutive amino acids of SEQ ID NO:1.


SEQUENCE LISTING (10 sequences)

SEQ ID NO: 1 - protein, 20 aa, Artificial Sequence
  Synthetically generated peptide
  VARIANT 5: Xaa = Ser or Pro
  VARIANT 6: Xaa = Gln or Pro
  VARIANT 7: Xaa = Ala or Thr
  VARIANT 12: Xaa = Asp or Asn
DVSTXXXVLPDXFPRYPVGK

SEQ ID NO: 2 - protein, 16 aa, Artificial Sequence
  Synthetically generated peptide
  VARIANT 5: Xaa = Ser or Pro
  VARIANT 6: Xaa = Gln or Pro
  VARIANT 7: Xaa = Ala or Thr
  VARIANT 12: Xaa = Asp or Asn
DVSTXXXVLPDXFPRY

SEQ ID NO: 3 - protein, 34 aa, Artificial Sequence
  Synthetically generated peptide
  VARIANT 5: Xaa = Ser or Pro
  VARIANT 6: Xaa = Gln or Pro
  VARIANT 7, 31: Xaa = Ala or Thr
  VARIANT 12: Xaa = Asp or Asn
  VARIANT 23: Xaa = Gln or Lys
  VARIANT 24: Xaa = Tyr or Phe
  VARIANT 28: Xaa = Arg or Lys
  VARIANT 32: Xaa = Gly or Gln
DVSTXXXVLPDXFPRYPVGKFFXXDTWXQSXXRL

SEQ ID NO: 4 - protein, 34 aa, Mus musculus
DVSTSQAVLPDDFPRYPVGKFFQYDTWRQSAGRL

SEQ ID NO: 5 - protein, 34 aa, Rattus norvegicus
DVSTSQAVLPDDFPRYPVGKFFKFDTWRQSAGRL

SEQ ID NO: 6 - protein, 34 aa, Homo sapiens
DVSTPPTVLPDNFPRYPVGKFFQYDTWKQSTQRL

SEQ ID NO: 7 - protein, 19 aa, Artificial Sequence
  Synthetically generated peptide
  VARIANT 5: Xaa = Ser or Pro
  VARIANT 6: Xaa = Gln or Pro
  VARIANT 7: Xaa = Ala or Thr
  VARIANT 12: Xaa = Asp or Asn
DVSTXXXVLPDXFPRYPVG

SEQ ID NO: 8 - protein, 18 aa, Artificial Sequence
  Synthetically generated peptide
  VARIANT 5: Xaa = Ser or Pro
  VARIANT 6: Xaa = Gln or Pro
  VARIANT 7: Xaa = Ala or Thr
  VARIANT 12: Xaa = Asp or Asn
DVSTXXXVLPDXFPRYPV

SEQ ID NO: 9 - protein, 17 aa, Artificial Sequence
  Synthetically generated peptide
  VARIANT 5: Xaa = Ser or Pro
  VARIANT 6: Xaa = Gln or Pro
  VARIANT 7: Xaa = Ala or Thr
  VARIANT 12: Xaa = Asp or Asn
DVSTXXXVLPDXFPRYP

SEQ ID NO: 10 - protein, 15 aa, Artificial Sequence
  Synthetically generated peptide
  VARIANT 5: Xaa = Ser or Pro
  VARIANT 6: Xaa = Gln or Pro
  VARIANT 7: Xaa = Ala or Thr
  VARIANT 12: Xaa = Asp or Asn
DVSTXXXVLPDXFPR